(19) United States
(12) Patent Application Publication
(10) Pub. No.: US 2010/0041034 A1
(43) Pub. Date: Feb. 18, 2010

(54) METHOD FOR MANIPULATING SAMPLES WITH MAGNETIC NUCLEATION NANOPARTICLES

(76) Inventors: Richard S. Murante, Rochester, NY (US); D. Michael Connolly, Rochester, NY (US)

Correspondence Address: HISCOCK & BARCLAY, LLP, 2000 HSBC PLAZA, 100 Chestnut Street, ROCHESTER, NY, 14604-2404, US

(21) Appl. No.: 12/423,539

(22) Filed: Apr. 14, 2009

Related U.S. Application Data
(60) Provisional application No. 61/044,841, filed on Apr. 14, 2008.

(51) Int. Cl. C12Q 1/68

(52) U.S. Cl. 435/6

(57) ABSTRACT

A method for manipulating a sample by utilizing a nucleic acid binding substance which has affinity for nucleic acid polymers. The nucleic acid binding substance is comprised of a nucleic acid binding element capable of specific binding to nucleic acid molecules and connected to a nucleation nanoparticle having paramagnetic properties.

### CROSS-REFERENCE TO RELATED APPLICATIONS

This application claims priority from U.S. Provisional Patent Application Ser. No. 61/044,841 , filed Apr. 14, 2008.

### FIELD OF THE INVENTION

This invention relates a method for manipulating samples by utilizing magnetic nucleation nanoparticles. In another embodiment the invention relates to a method for concentrating a sample by utilizing magnetic nucleation nanoparticles.

### BACKGROUND OF THE INVENTION

In running biological and chemical tests it is often desired to concentrate a sample to retain desired analyte. Concentrating the sample can be a difficult process. Traditional methods for concentrating a biological sample include filtering, rinsing, centrifuging and/or reaction chemistry. Often these steps cannot be preformed in a single processing chamber and require the sample to be transferred to other devices or chambers.

Paramagnetic particles are particles which are attracted to a magnetic field. Unlike ferromagnetic particles, paramagnetic particles retain little or no magnetic properties in the absence of a magnetic field. By attaching a paramagnetic nucleation particle to nucleic acid polymers and applying a magnetic field to a sample, the nucleic acid polymers can be moved to a desired location, thereby concentrating a portion of the sample with the nucleic acid polymers. The sample can then be drawn from the concentrated portion yielding a high amount of nucleic acid polymers.

Appling a magnetic field further allows for manipulating the nucleic acid polymer into distinct chambers at speeds faster than the diffusion rate. Additionally, by holding a nucleic acid polymer steady a rinse can be applied without washing away the nucleic acid polymer.

In array situations applying a magnetic field allows for positioning the nucleic acid polymer in the vicinity of a desired test area. The nucleic acid polymer can be manipulated to sequentially interact with a plurality of test areas.

Therefore, a magnetic nucleation particle that specifically binds to target analytes is desired.

Further a nucleation particle having paramagnetic properties is desired.

### SUMMARY OF THE INVENTION

The invention comprises, in one form thereof, a method for utilizing magnetic nucleation nanoparticle containing a target analyte binding element to bind the nucleation nanoparticle to a target analyte. The magnetic nucleation nanoparticle is capable of being manipulated within a magnetic field. As the magnetic nucleation nanoparticle is attached to the target analyte the target analyte is indirectly manipulated by the application of a magnetic field.

In one form, the target analyte binding element links directly to the particle surface. Optionally, the target analyte binding element is attached to the magnetic nucleation nanoparticle via intermediate connecting groups such as, but not limited to linkers, scaffolds, stabilizers or steric stabilizers. The intermediate connecting group can be of variable size, architecture and chemical composition to interconnect the magnetic nucleation nanoparticle(s) and the target analyte binding element(s) into a multifunctional entity. In another embodiment the magnetic nucleation nanoparticle further contains a catalytic material.

In one embodiment, the target analyte binding group functionalized particle require improved colloid stability to prevent agglomeration. Therefore, a colloid stabilizer, such as a hydrophilic chain or ionic group, is added or connected to a linking group that links to the particle. These groups assist in limiting the nanoparticles size during the particle generation stage.

An advantage of the present invention is that the utilization of magnetic nucleation nanoparticles allows for sample concentration by applying a magnetic field without additional processing steps.

A further advantage of the present invention is that the utilization of magnetic nucleation nanoparticles allows for rapid manipulation of target analytes thereby reducing diffusion and reaction times.

### BRIEF DESCRIPTION OF THE DRAWINGS

The present invention is disclosed with reference to the accompanying drawing, wherein:

FIG. 1 is a graph showing the binding percentage of DNA molecules as a function of the fraction of ethanol in the mixture.

The examples set out herein illustrate several embodiments of the invention but should not be construed as limiting the scope of the invention in any manner.

### DETAILED DESCRIPTION

Magnetic nucleation nanoparticles located in a sample chamber along with a target analyte. The magnetic nucleation nanoparticles have an affinity for the target analyte. By attaching the magnetic nucleation nanoparticles to the target analyte and applying a magnetic field the target analyte is manipulated to desired locations within the sample chamber.

In one embodiment, the target analyte binding element is attached to the magnetic nucleation nanoparticle via at least one intermediate connecting group such as, but not limited to linkers, scaffolds, stabilizers or steric stabilizers.

The nucleation nanoparticle contains particles that exhibit paramagnet properties. There are a number of particles that exhibit paramagnetic properties. In one embodiment cobalt, nickel, iron or a combination thereof is used to create a paramagnetic nucleation nanoparticle. Optionally, the paramagnetic nucleation nanoparticle further contains a catalytic particle. In one embodiment the catalytic particle is palladium, platinum, silver or gold.

In another embodiment, the nucleation nanoparticles contain ferrimagnetic particles. The ferrimagnetic particles include metal oxides, such as iron oxides. Suitable iron oxides include hematite (Fe₂O₃) and magnetite (Fe₃O₄).

In one form, a nickel-palladium nanoparticle, stabilized by a surface layer of 4-dimethylaminopyridine as described in Flanagan et al, Langmuir, 2007, 23, 12508-12520, is treated by adsorption with a plurality of ethidium bromide intercalator molecules to create nucleic acid binding sites. The ethidium moiety bonds to the nucleic acid polymer thereby attaching the nickel-palladium nanoparticle to the nucleic acid polymer.

In another form, a simple straight-chain scaffold molecule, such as oligoethylene glycol (PEG), is affixed with a nucleic acid binding element at one end and a linker at the other end. The nucleic acid binding element binds to the nucleic acid polymer and the linker binds to the paramagnet nucleation nanoparticle. The nucleic acid binding element is an intercalator, such as ethidium bromide, or a minor groove binder such as distamycin. The linker is a phenanthroline derivative. Hainfeld, J. Structural Biology, 127, 177-184 (1999) reports the advantage of phenanthroline derivatives in creating palladium particles. The scaffold may be a simple difunctional straight chain as shown, or may be a multifunctional branched scaffold connecting multiple catalytic nucleation nanoparticles or nucleic acid binding elements. The nucleic acid binding element bonds to the nucleic acid polymer, thereby attaching the nanoparticle to the nucleic acid polymer. It is understood that additional nucleic acid binding elements and intermediate connecting groups are within the scope and may be used.

### Concentration of Target Analyte:

The sample containing the target analyte is located in a reaction chamber. The reaction chamber contains both the sample and magnetic nucleation nanoparticles. The magnetic nucleation nanoparticles bind to the target analyte. In one embodiment the reaction chamber further contains disrupting beads to assist in breaking apart samples to provide access to the target analyte.

Once the sample is lysed, the nucleic acid molecules can be magnetically separated from the reminder of the sample. The nucleic acid molecules bind to magnetic particles. In one embodiment, the binding occurs in a high salt/ethanol conditions and is eluted using a low salt buffer with increased temperature. In one embodiment the sample is heated to at least 95° C. to increase yield from elution.

Once the magnetic nucleation nanoparticles are attached to the target analyte a magnetic field is applied to the reaction chamber. The application of the magnetic field causes the magnetic nucleation nanoparticles and any attached target analytes to concentrate in one portion of the reaction chamber. The sample is pulled from the concentrated region of the sample chamber providing a large amount of target analytes comparative the amount of volume extracted. By concentrating the sample more sensitive tests can be preformed.

In another embodiment, the magnetic field holds the magnetic nucleation nanoparticle steady as the remaining sample is removed from the chamber. The binding force between the magnetic nucleation nanoparticle and the target anaylte is sufficient to prevent the target anaylte from being removed. Optionally, additional rinse steps are used to purify the sample.

### Rapid Movement and Increased Sensitivity:

Typically in solution a target analyte is limited in movement by fluid flow and diffusion rates. To speed the movement of a target analyte through the system a magnetic field is applied to progress the magnetic nucleation nanoparticle to the desired location. The application of the magnetic field allows for rapid transport of the target anaylte from one chamber to another.

An array of sensors are used to rapidly detect the target analyte. A magnetic field is applied to guide the magnetic nanoparticles and attached analytes to the vicinity of a first sensor. A distinct magnetic field then guides the magnetic nanoparticles and any attached target analytes to a second senor. The magnetic field is manipulated to move the target analytes to each sensor in the array. In one embodiment, the sensor binds a particular target analyte with enough force to prevent the magnetic field from breaking the bond. By systematically applying magnetic fields the analysis time is greatly reduced compared to normal diffusion analysis.

### Magnetic Nucleation Nanoparticles:

Use of sols or clusters in the form of magnetic nucleation nanoparticles allows for the attachment of paramagnetic material to a target nucleic acid polymer or other target analyte. By applying a magnetic field to the sample the nucleic acid polymer can be manipulated via the attached paramagnet material.

The paramagnet nucleation nanoparticles are formed in solution with a stabilizer. In one embodiment a metal salt is used. A reducing agent, such as imethylamineborane or sodium borohydride, is added to the solution. If needed, solvents and excess salts can be removed by centrifugation, decantation, washing, and resuspension of the metal clusters. Alternatively, a magnetic field can be applied to the solution holding the paramagnetic nucleation nanoparticles in place as a drain and rinse is applied.

### Target Analyte Binding Element:

The target analyte binding element attaches to the magnetic nucleation nanoparticle, either directly or by way of an intermediate connecting group. The target analyte binding element further binds to the nucleic acid polymer. In one embodiment the target analyte binding element is a nucleic acid binding element such as a molecule, fragment or functional group that binds to nucleic acid polymers. Potential nucleic acid binding elements consist of intercalators, minor groove binders, cations, amine reactive groups such as aldehydes and alkylating agents, proteins, and association with hydrophobic groups of surfactants. In addition, functional groups such as aldehydes are used to create a connection by reaction with free amines in the nucleic acid. Other amine reactive groups such as Michael addition are suitable.

Examples of structures that form the basis for intercalating and minor groove binder structures are:

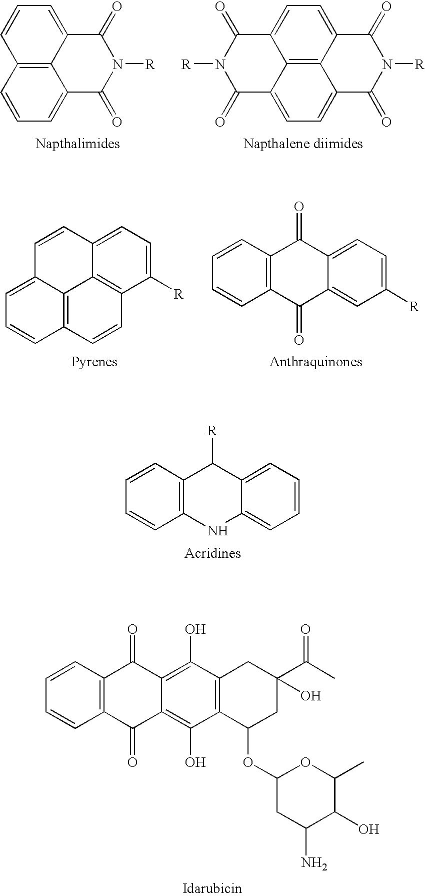

The range of specific intercalator and minor groove binder structures is enormous as the field has been the subject of intense study for over 50 years. See R. Martinez and L Chacon-Garcia, Current Medicinal Chemistry, 2005, 12, 127-151. Therefore, the R groups include a broad range of organic functional groups. In many cases, interaction can be enhanced if R contains hydrogen bonding, cationic or hydrophilic character.

In addition, compounds such as cationic polymers, such as polyethyleneimine, interact with nucleic acid and have been proposed as gene carriers as evidenced by Xu et al, International Journal of Nanoscience, 2006, 5, 753-756 and Petersen et al, Bioconjugate Chemistry, 2002, 13, 845-854. Proteins are another well known class of materials that offer useful nucleic acid interaction and could be the basis for attaching nanoparticles to nucleic acids. Direct reaction with functional groups on the nucleic acid is also within the scope of this invention. For example, amine groups can be reacted with aldehydes to create a bond (Braun et al, Nano Letters, 2004, 4, 323-326)

In one embodiment the nucleic acid binding elements are specific binding agents that specifically target double-stranded nucleic acid molecules while not binding with single-stranded nucleic acid molecules. For example, minor-groove binding compounds specifically bind hybridized double-stranded DNA molecules, but do not bind to single-stranded oligonucleotide capture probes. In contrast, palladium chloride reagent indiscriminately binds to both the target molecules and capture probes. The binding element binds specifically to the target nucleic acid molecule while having little or no affinity towards non-target molecules. It is understood that the specific binding elements can include but are not limited to intercalators, minor-groove binding compounds, major-groove binding compounds, antibodies, and DNA binding proteins. The specific binding element binds to a specific site on a target nucleic acid without binding to non-desired areas. In one embodiment, the specific binding element is ethidium bromide. In alternative embodiments, the specific binding element is distamycin, idarubicin, or Hoescht dye.

In one embodiment the nucleic acid binding element also serves as a stabilizer as described below.

### Stabilizers:

The magnetic nucleation nanoparticles are surface functionalized with stabilizers to impart desirable properties. These stabilized nucleation nanoparticles demonstrate colloid stability and minimal non-specific binding. Furthermore, the presence of the stabilizer in solution while forming the paramagnetic nucleation nanoparticle controls the nanoparticle size.

The stabilizer provides colloid stability and prevents coagulation and settling of the magnetic nucleation nanoparticle. The stabilizer further serves to limit the size of the paramagnetic nucleation nanoparticle during the formation process. In one embodiment, metal paramagnetic nucleation nanoparticle are formed in a solution containing stabilizer and metal ions. In one embodiment the stabilizers are chelating compounds. Large paramagnetic nucleation nanoparticles are undesirable as they are more likely to precipitate out of solution. Therefore, the nucleation nanoparticle shall be small enough to remain in solution. In one embodiment, the nucleation nanoparticle is generally spherical in shape with a diameter from about 0.5-100 nm. Preferably, the nucleation nanoparticle is generally spherical in shape and has a diameter from about 1-100 nm.

Suitable stabilizers include, but are not limited to, polyethyloxazoline, polyvinylpyrollidinone, polyethyleneimine, polyvinylalcohol, polyethyleneglycol, polyester ionomers, silicone ionic polymers, ionic polymers, copolymers, starches, gum Arabic, suractants, nonionic surfactants, ionic surfactants, fluorocarbon containing surfactants and sugars. In one embodiment the stabilizer is a phenanthroline, bipyridine and oligovinylpyridine of the following formulas:

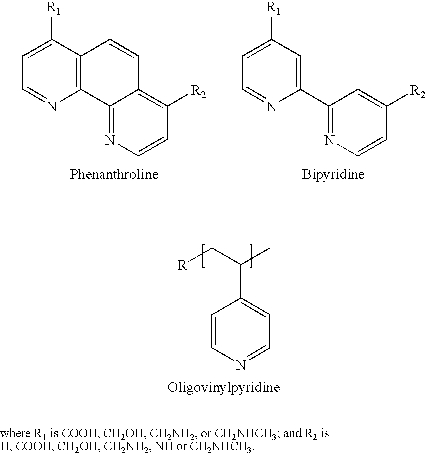

In one embodiment where the paramagnetic nucleation nanoparticle contains palladium, these stabilizers link by acting as ligands for palladium ions and are therefore closely associated with the particle formation. In addition to linking, the stabilizers have hydrophilic groups that interact with the water phase. The linking and stabilization function of molecules such as phenathrolines in palladium particle formation is further described in Hainfeld, J. Structural Biology, 127, 177-184 (1999).

It is understood that particles derived from a broad class of materials plastics, pigments, oils, etc) in water can be stabilized by a wide array of surfactants and dispersants that don't rely on specific coordination. These classes of stabilizers are also within the scope of this invention.

In one embodiment the stabilizer stabilizes the paramagnetic nucleation nanoparticle from precipitation, coagulation and minimizes the non-specific binding to random surfaces. In another embodiment, the stabilizer further functions as a nucleic acid binding element as described below.

### Linker:

The linker is bound directly to the magnetic nucleation particle to allow the attachment of other intermediate connecting groups or target analyte binding elements. It is understood that the linker can also serve as a stabilizer or scaffold.

The linker can be bound through various binding energies. The total binding energy consists of the sum of all the covalent, ionic, entropic, Van der Walls and any other forces binding the linker to the catalytic nucleation nanoparticle. In one embodiment, the total binding energy between the linker and the paramagnetic nucleation particle is greater than about 10 kJ/mole. In another embodiment the total binding energy between the linker and the paramagnetic nucleation particle is greater than about 40 kJ/mole. Suitable linkers include, but are not limited to ligands, phenanthrolines, bidentates, tridentates, bipyridines, pyridines, tripyridines, polyvinylpyridines, porphyrins, disulfides, amine acetoacetates, amines, thiols, acids, alcohols and hydrophobic groups.

### Scaffold Compositions:

The paramagnetic acid binding element may be connected directly to the catalytic nucleation particle or a linker. Alternatively, the nucleic acid binding element is attached to a scaffold, either individually or as a multiplicity. In either case, the final conjugate is endowed with the two essential properties—nucleic acid specific recognition-binding and an attached paramagnetic nucleation nanoparticle. Attaching the nucleic acid binding element to the scaffold may be by way of any of the common organic bonding groups such as esters, amides and the like.

Attachment to a common scaffold creates an enormous range of possible sizes, shapes, architectures and additional functions. In one embodiment the scaffold composition is a linear chain with the two functional groups at the ends. The chain itself can be of any composition, length and ionic character. In an alternative embodiment, often used in biological applications, polyethylene glycol with a reactive amine, acid or alcohol end groups is utilized as included in the following example.

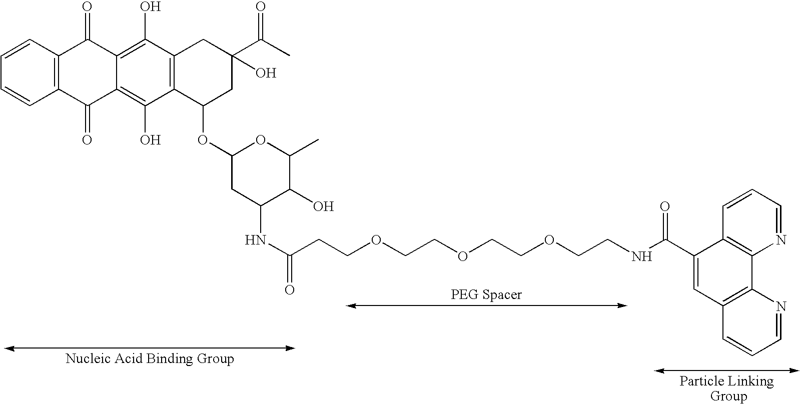

Linear short spacers with cationic character can be desirable as they can enhance intercalation performance.

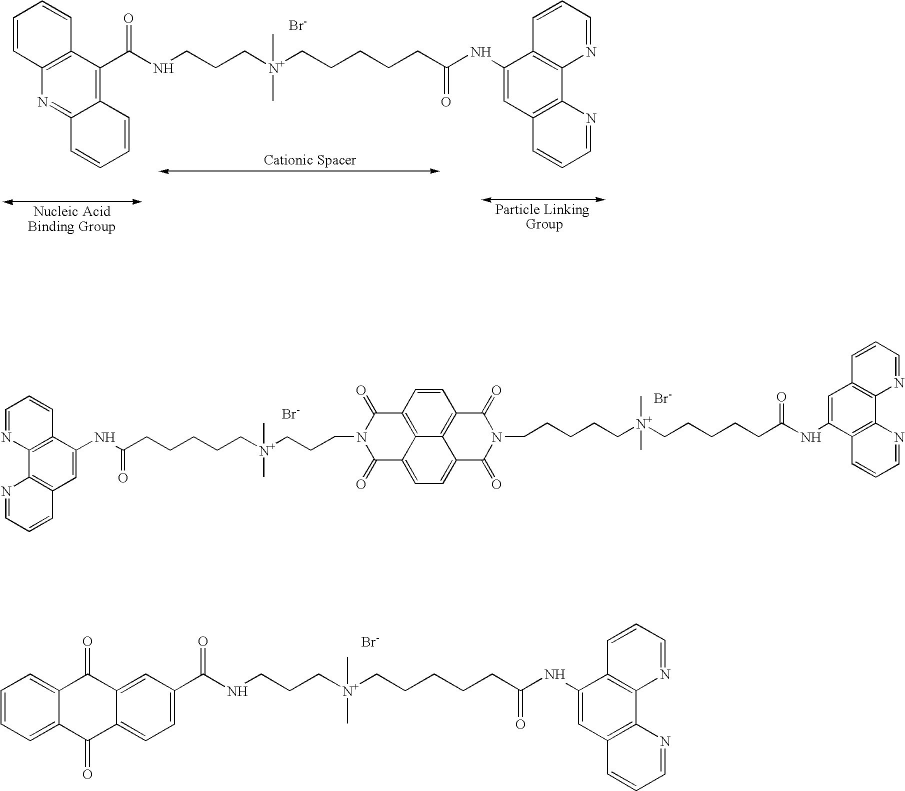

A polymeric or oligomeric scaffold allows for multiple groups to be joined in the same structure where the number of groups is limited only by the size of the chain.

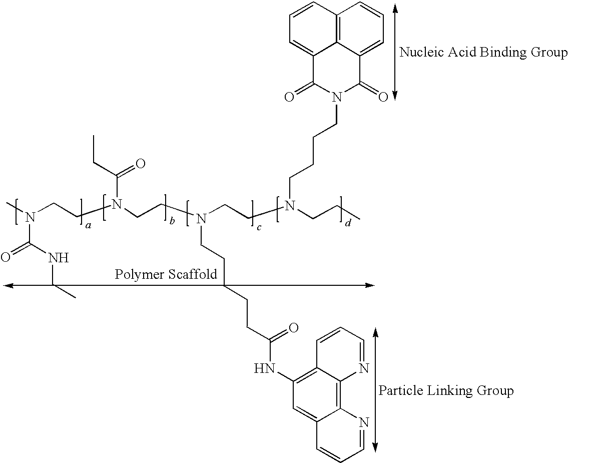

In addition to short and long chain structures scaffolds can be built with branched or very highly branched architectures. Furthermore, scaffolds can be a microgel particle with nanoparticles bound to a swollen polyvinylpyridine interior and peripheral nucleic acid binding elements are illustrated. In another embodiment the scaffold is a core-shell latex particle with nucleation nanoparticles centers and peripheral nucleic acid recognition groups populating the surface. It is understood that any scaffold compositions can be incorporated to connect intermediate connecting groups, catalytic nucleation nanoparticles or nucleic acid binding elements.

### Steric Stabilizers:

In one embodiment a steric stabilizer is used to attach the target analyte binding element to the paramagnetic nucleation nanoparticle. The steric stabilizer is capable of functioning as a stabilizer, linker and scaffold as described above. In one embodiment the steric stabilizer is polyethylenimine, polyethyloxazoline or polyvinylpyrrolidone. The steric stabilizer binds to the paramagnetic nucleation particle with a total binding energy of at least 10 kJ/mole. In another embodiment the steric stabilizer binds to the paramagnetic nucleation particle with a total binding energy of at least 40 kJ/mole. The use of steric stabilizers eliminate any need for distinct stabilizers, linkers, or scaffolds. One or multiple nucleic acid binding elements can be attached to the steric stabilizer. Furthermore, one or multiple paramagnetic nucleation nanoparticles can be bound to the steric stabilizer.

### Target Analyte Binding Substance:

In one embodiment for forming the target analyte binding substance on a nucleation nanoparticle, the nucleation nanoparticles are formed in solution with a stabilizer such as dimethyaminopyridine (DMAP). The stabilized nucleation nanoparticles are purified to retain clusters of the desired size. The nanoparticles are then treated directly with a nucleic acid binding element such as ethidium bromide or with a nucleic acid binding element connected to a linker or with a scaffold composition containing the nucleic acid binding element. The scaffold composition can be a polymer containing nucleic acid binding elements such as napthalimide or acridine. The polymer displaces some of the DMAP and attaches to the particle. It is understood that the nucleic acid binding element can be chemically attached to the scaffold composition prior to the attachment of the scaffold composition to the particle.

In another embodiment for forming the target analyte binding substance on a nucleation particle, the nucleation nanoparticles are formed in solution in the presence of a nucleic acid binding element such as ethidium bromide or in the presence of a nucleic acid binding element connected to a linker or in the presence of a scaffold composition containing the nucleic acid binding element. The scaffold composition can be a polymer containing nucleic acid binding elements such as napthalimide or acridine. It is understood that the nucleic acid binding substance connects to the particle during the particle formation process and may offer some colloidal stability to the dispersion. In addition, stabilizers in the form of ionic surfactants, non ionic surfactants, water soluble oligomers and polymers may also be added to enhance colloid stability and control particle size.

### PARAMAGNETIC EXAMPLES

Metal salts (nickel, cobalt, iron) with a small amount of palladium salt are dissolved in a solvent (water and/or polar organic solvent) along with a stabilizer (phenanthroline, bipyridine, polyvinylpyrrolidinone). A reducing agent is added (dimethylamineborane, sodium borohydride) and the mixture is held until the metal clusters are formed. If needed, solvents and excess salts can be removed by centrifugation, decantation, washing, and resuspension of the metal clusters.

Solution A—24 g of nickel chloride hexahydrate and 44 g of sodium citrate were dissolved in 500 ml of water.

Solution B—24 g of ethanolamine were dissolved in 500 ml of water.

Solution C—5 g of cobalt chloride hexahydrate were dissolved in 100 ml water.

Solution D—2 g of potassium tetrachloropallidate and 6 g of potassium chloride were dissolved in 100 ml of water.

Solution E—1 g of bathophenanthroline-disulfonic acid, disodium salt hydrate was dissolved in 100 ml water.

Solution F—3 g of dimethylamine borane were dissolved in 100 ml water.

### EXAMPLE 1

In a 20 ml glass vial, 1 ml solution A and 1 ml of solution B were mixed. 0.1 ml of solution D was added, followed immediately by 0.2 ml of solution E. Then 0.5 ml of solution F was added and the mixture was held at 60 degrees C. for 30 minutes. After cooling to room temperature, the mixture was placed in a strong magnetic field for 10 seconds (the magnetic field was from the permanent magnetic removed from a discarded computer hard drive) and it was observed that most of the metal clusters moved to the wall of the vial nearest the magnet.

### EXAMPLE 2

In a 20 ml glass vial, 0.2 ml solution A, 0.8 ml solution C and 1 ml of solution B were mixed. 0.1 ml of solution D was added, followed immediately by 0.2 ml of solution E. Then 0.5 ml of solution F was added and the mixture was held at 60 degrees C. for 30 minutes. After cooling to room temperature, the mixture was placed in a strong magnetic field for 10 seconds (the magnetic field was from the permanent magnetic removed from a discarded computer hard drive) and it was observed that most of the metal clusters moved to the wall of the vial nearest the magnet.

### Preparation of Magnetite Clusters:

### EXAMPLE 3

A first solution of ferric chloride (0.8M), ferrous chloride (0.4M) and hydrochloric acid (0.4M) was mixed and 0.2 micron filtered. A second solution was prepared with 72 ml of ammonium hydroxide (30%) with water to make 1 liter.

1 ml of the ferric/ferrous chloride solution was added with stirring to 20 ml of the ammonium hydroxide solution. Stirring was continued for 15 seconds. The solution (in a 20 ml vial) was placed on a strong magnet and allowed to stand for 1 minute, after which all the product was pulled to the bottom of the vial. The clear supernatant liquid was decanted, replaced with water, mixed, and placed near the magnet. Again the product was pulled to the bottom of the vial. This process was repeated three times to wash the product free from any residual ammonium and iron salts. The vial was then filled with 20 ml of water and ultra-sonicated for 5 minutes at 4 watts power. The suspension was then filtered through a 1 micron glass filter to give a stable suspension of magnetite particles that remain in suspension until pulled down by magnetic forces or centrifugation.

### Attachment of Magnetic Particles:

### EXAMPLE 4

Nucleic acid molecules were purified from fruit flies, then lysed with ferrous particles followed by magnetic separation and elution. The magnetic beads captured more than 90% of available nucleic acid molecules.

### Hybridizing to Capture Probes:

### EXAMPLE 5

Once the nucleic acid molecules are prepared, they are hybridized to capture probes on sensor electrodes. Samples of nucleic acid molecules from Bacillus cells were prepared through ultrasonic lysis and magnetic concentration. The eluted DNA was bound to probes on the sensor chip to demonstrate that there are no inhibitors of hybridization.

### Sample Cleaning:

In one embodiment, the sample is cleaned to remove compounds which could potentially inhibit the binding of nucleic acid molecules to sensors. By attaching magnetic particles to the sample and manipulating the sample with a magnetic field the sample is both concentrated and cleaned from impurities.

### EXAMPLE 6

Bacterial and spore samples mixed with soil were processed to evaluate complex samples. Soil is a complex medium which is known to inhibit PCR-based systems. Soil was added to samples containing six whole fruit flies. The flies are intended to represent insects that might be evaluated for carrying a disease like malaria. Up to 320 micrograms of the soil were added per milliliter of sample. The fruit flies were lysed and the DNA and RNA were captured using ferrite particles with the addition of ethanol. The particles were collected magnetically, washed with buffer and ethanol to remove contaminants then concentrated with magnetics. The nucleic acid molecules were then eluted in hybridization buffer at 90° C. to denature the DNA component. The ferric particles worked well in the presence of soil. Minimal loss was seen until the level of soil in the sample reached 32 milligrams per 100 micro liters where the solution becomes viscous and particle movement is difficult.

DNA from Complex Samples:

### EXAMPLE 7

Bacillus cells were mixed with cattle ear tissue or whole fruit flies and the mixtures were taken through the sample preparation process. The resulting nucleic acids were hybridized to probes on sensor chips. The chips were then treated with YOYO-1 dye to detect hybridized DNA. The target DNA sequences in the cells hybridized to the sensor chips at levels comparable to Bacillus cells processed separately. Negative controls without Bacillus showed no hybridized DNA. The experiment was repeated with dirt added to the samples as described above. Hybridization efficiency remained at least 60% of the hybridization seen in the sample without eukaryotic cells and dirt.

Washing Particles with a Flow:

### EXAMPLE 8

Magnetic particles were bound to DNA and then the solution introduced into a clear plastic tube with a 2 mm diameter. A magnet was placed under the center of the tube. A wash buffer was pushed through the tube using a syringe pump. The particles visually remained in place through the washing. After washing the magnet was removed and the particles were rinsed out of the tube. DNA was eluted at high temperature and run on a gel. No apparent loss of DNA was observed.

### Efficiency of Binding and Release of Magnetic Particles:

### EXAMPLE 9

Radiolabled DNA was used to determine the efficiency of binding to ferrite and the release of the nucleic acid molecules. Radiolabeled DNA with the magnetite suspension and three volumes of ethanol were mixed. The magnetite was pulled to the bottom of the tube using a magnet. The supernatant fluid was removed from the pellet and both fractions were counted in a scintillation counter. The supernatant contained 770 cpm and the resuspended pellet contained 19,330 cpm. Therefore about 96% of the Radiolabled DNA was bound to the ferrite.

### EXAMPLE 10

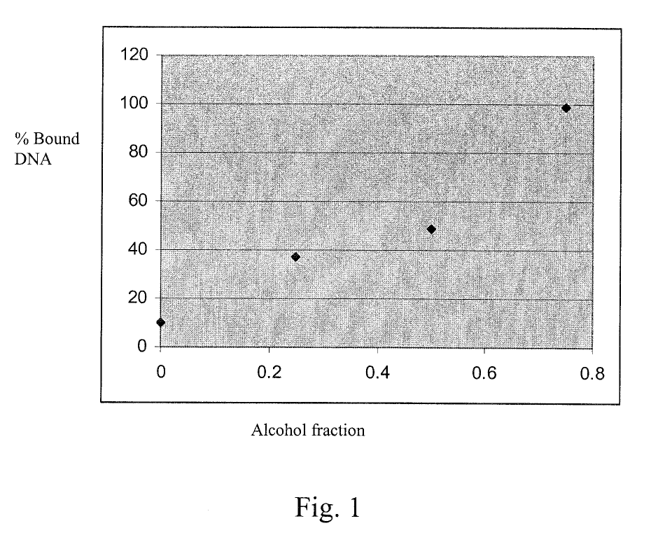

Radiolabled DNA was used to determine the efficiency of binding to ferrite and the release of the nucleic acid molecules. Radiolabeled DNA with the magnetite suspension and three volumes of ethanol were mixed. The magnetite was pulled to the bottom of the tube using a magnet. The supernatant fluid was removed from the pellet and both fractions were counted in a scintillation counter. Binding was measured as a function of the fraction of ethanol in the mix. The results are plotted in FIG. 1.

To determine the release efficiency, the bound DNA pellet is suspended in 100 μl of buffer as indicated in the table below, incubated for 10 minutes at 95° C., then collected on the magnet. The supernatant was separated from the pellet and both were counted.

| Buffer | Supernatant cpm | Pellet cpm | % Free |
|---|---|---|---|
| 500 mM Phosphate | 43,450 | 1925 | 96% |
| 50 mM Phosphate | 18,409 | 684 | 96% |
| 60 mM Citrate | 33,276 | 2164 | 94% |
| 100 mM Tris | 911 | 35,878 | 3% |
| 0.2% SDS | | | |
| 1.5% Dextran sulfate | | | |

The Tris buffer with SDS can be used for hybridization with magnetite bound DNA in order to allow for magnetic concentration of DNA or RNA near the sensor.

### Rapid Movement of Particles:

### EXAMPLE 10

Microchips were fabricated with metal coils having line widths of one micron. A current was run through the coils to produce a magnetic field. A solution containing magnetic nano-particles was then spotted over the coils. The chip was placed under a microscope and current turned on through the coil. Within 10 seconds, clusters were congregating at the corners within the coil. Once the current was turned off the particles demagnetize and begin to diffuse back into solution.

While the invention has been described with reference to particular embodiments, it will be understood by those skilled in the art that various changes may be made and equivalents may be substituted for elements thereof without departing from the scope of the invention. In addition, many modifications may be made to adapt a particular situation or material to the teachings of the invention without departing from the scope of the invention.

Therefore, it is intended that the invention not be limited to the particular embodiments disclosed as the best mode contemplated for carrying out this invention, but that the invention will include all embodiments falling within the scope and spirit of the appended claims.

1. A method for manipulating a target analyte comprising the steps of:
attaching a magnetic nucleation nanoparticle to the target analyte; and
applying a magnetic field strong enough to manipulate said magnetic nucleation nanoparticle.

2. The method of claim 1 wherein said magnetic nucleation nanoparticle is paramagnetic.

3. The method of claim 1 wherein the target analyte is a nucleic acid polymer.

4. The method of claim 3 wherein the nucleic acid polymer is DNA.

5. The method of claim 1 wherein the nucleation nanoparticle is generally spherical in shape and has a diameter from about 1-100 nm.

6. A method for concentrating a sample comprising the steps of:
attaching a paramagnetic nucleation nanoparticle to a target analyte within a sample chamber;
applying a magnetic field to the sample chamber causing the paramagnetic nucleation nanoparticles to concentrate in a first portion of the sample chamber; and
drawing sample from the first portion of the sample chamber.

7. The method of claim 6 further comprising the step of agitating a plurality of beads causing the beads to disrupt and break apart large sample structures.

8. The method if claim 6 wherein the target analyte is a nucleic acid polymer.

9. The method of claim 8 wherein the nucleic acid polymer is a double stranded DNA molecule.

10. A method for manipulating a target analyte within an array comprising the steps of:
attaching a magnetic nucleation nanoparticle to a target analyte;
applying a first magnetic field causing the magnetic nucleation nanoparticle and target analyte to concentrate at a first test site;
removing the first magnetic field; and
applying a second magnetic causing the magnetic nucleation nanoparticle and target analyte to concentrate at a second test site.

11. The method of claim 10 wherein the target analyte is double stranded DNA molecule.

12. The method of claim 10 wherein the first test site is a set of oligonucleotide probes separated by a gap.
